(19) United States
(12) Patent Application Publication
(10) Pub. No.: US 2011/0130372 A1
(43) Pub. Date: Jun. 2, 2011

(54) COMPOSITION COMPRISING ESTRONE AND HYDROCORTISONE FOR USE IN THE TOPICAL TREATMENT OF BALDNESS

(76) Inventors: Gaetano Agostinacchio, Porto Recanati (IT); Andrea Marliani, Firenze (IT); Marino Salin, Barberino Val D'elsa (IT)

(21) Appl. No.: 13/003,897

(22) PCT Filed: Jul. 24, 2009
(86) PCT No.: PCT/IB2009/006343

§ 371 Date: Jan. 27, 2011

(30) Foreign Application Priority Data
Jul. 29, 2008 (IT) MI2008A001401

(51) Int. Cl. A61K 31/57; A61K 31/573; A61P 17/14

(52) U.S. Cl. 514/170; 514/179

(57) ABSTRACT

The present invention relates to a composition comprising an association of active substances for use in the topical treatment of baldness, particularly of male pattern and female pattern androgenetic alopecia.

The present invention relates to a composition comprising an association of active substances for use in the topical treatment of baldness, particularly of male pattern and female pattern androgenetic alopecia. An excessive hair loss is a very common condition among the world population and can involve both sexes, although it affects in particular the male population.

In its first stages, this condition leads to a thinning and then to a real hair loss, i.e. the absence of hair in skin areas normally characterized by their presence, a condition which is normally referred to as baldness.

As is known, hair cycle is controlled by sexual steroid hormones, particularly not by circulating hormones but by hormones produced in situ by follicles.

There are two essential intrafollicular hormones for the regulation of hair cycle: dihydrotestosterone and estrone. Dihydrotestosterone leads the follicle to catagen and the hair to telogen. Estrone maintains matrix mitosis and anagen duration, and activates stem cells at the beginning of anagen.

The well-known clinical picture of androgenetic alopecia is related to the activity of 5-alpha-reductase and of dihydrotestosterone. It is now widely accepted that this is due to a genetic message which needs male hormones to manifest itself (Hamilton). In other words, the genotype (baldness inheritance) becomes phenotype (clinical manifestation of baldness) in the presence of androgenic hormones only.

Androgenetic alopecia is supported by the presence of normal androgenic hormones in plasma, by a familiar multigenic inheritance (whence the term “androgenetic”) and by the activity in hair follicles of enzymes that are able to convert steroids into hormones acting towards the follicle. In particular, a crucial point is represented by the activity of the enzyme 5-alpha-reductase, which converts testosterone into di-hydrotestosterone (DHT).

Conversely, the Applicants believe that the clinical picture of diffused alopecia known as deficiency alopecia (low local estrone hypotrichosis) is related to a reduced activity of aromatase and/or of 3-alpha-reductase. In women, except for some rare cases of abnormal adrenal or ovarian hormone production due to an enzymatic defect or a secreting tumour, alopecia appears in quite a different form with respect to men and mechanisms are also different and, although they have not been wholly explained yet, they can almost always be related to a local estrone deficiency.

The cases of girls with thin hair on their whole scalp (though more on the vertex and in the front area) whose mothers are (often) in the same conditions, though with normal menses and fertility, without an excess of circulating androgenic hormones and for whom no clear lab or clinical evidence of telogen effluvium can be found, suggest a familiar peripheral resistance of the follicle to the action of estrogens (deficiency of 17-steroid-oxidoreductase, aromatase, 3-alpha-reductase). Those are therefore cases of deficiency alopecia.

Eventually, a clinical picture of front-parietal alopecia (the so-called male receding hairline) can be related to the direct activity of testosterone. In order to understand front-parietal alopecia, it is important to take into consideration the progress of hair loss in men and women.

Men experience first a recession of the front hair line with hair thinning on the vertex, then hair thinning on the temples, which gives the hair cut the characteristic male M shape.

Up to this point male hair loss can be regarded as physiologic and not necessarily as a sign of baldness, that is why this is preferably referred to as “male front-parietal alopecia”.

Considering that there are men with receding hairline but not bald, and bald men but not with receding hair-line, and that 5-alpha-reductase inhibitors do not reduce in any way the speed of appearance of front-parietal alopecia, one may assume that the latter is induced by testosterone, whereas (real) baldness is induced by its main metabolite, dihydrotestosterone.

In male baldness the vertex slowly gets “empty” and progressively joins the “bald” front-parietal areas, first leaving a kind of “island” untouched above the forehead and eventually achieving “crown baldness”. Generally, defluvium then becomes stable leaving temple and occipital areas untouched, and the process stops.

Hair loss in the front-parietal area and the receding hairline are due to the direct action of testosterone, whereas real androgenetic alopecia involves the vertex and is due to the action of dihydrotestosterone. That is why all men have a more or less receding hairline and a M-shaped hair cut is physiological for men just like beard growing.

Today there is no medical therapy for advanced baldness which, though refined, can be really beneficial to the patient. Baldness can only be partially solved by re-distributing hair with surgical techniques. The techniques used at present are just improvements of three traditional methods: scalp reduction, the so-called “punch-graft transplantation” or Orentreich technique and “flap rotation” or Juri technique.

As far as the medical therapy is concerned, it is generally followed and monitored by trichological examinations and is stopped only when trichograms show normal ratios between cycle phases (anagen-catagen-telogen) and the examination of fallen hair shows a sufficiently low value of “premature telogen phases” (<6%). Trichological examinations are however repeated at periodic intervals (every 6-12 months) so as to immediately identify any onset of defluvium.

There is therefore a strong need for medical treatments that are able to reduce and/or stop hair loss and that are also able to induce hair re-growth without unwanted collateral effects.

After long and in-depth clinical studies, the Applicants have found that compositions comprising an association of active substances as defined below are particularly useful in the topical treatment of baldness, particularly of male pattern androgenetic alopecia and of female pattern, so-called androgenetic, alopecia.

Therefore, in a first aspect the present invention relates to a composition comprising:
(a) from 0.005 to 0.2% by weight of estrone;
(b) from 0.005 to 0.2% by weight of hydrocortisone or an ester thereof or a pharmaceutically acceptable salt thereof,
   for use in topical treatment of baldness.

Preferably, the composition according to the present invention comprises:
(a) from 0.01 to 0.1% by weight of estrone;
(b) from 0.01 to 0.1% by weight of hydrocortisone or an ester thereof or a pharmaceutically acceptable salt thereof.

Compositions of this type are particularly useful in the treatment of baldness for female patients.

If the treatment is designed for male patients, the composition according to the invention further comprises:
(c) 0.02 to 6% by weight, preferably 0.5 to 4% by weight, of progesterone.

As is known, estrone is an estrogenic hormone, secreted by the fatty tissue, by the ovary, by the testicle and by the adrenal gland, having the following formula:

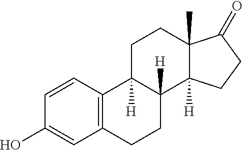

IUPAC name: 3-hydroxy-13-methyl-6,7,8,9,11,12,13,14, 15,16-decahydrocyclopenta[a]phenanthren-17-one.

As is known, hydrocortisone is a corticosteroid produced by the adrenal gland, which can also be obtained by synthesis, having the following formula:

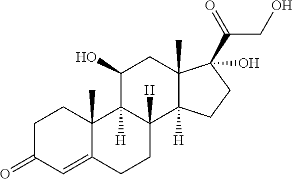

IUPAC name: 11,17,21-trihydroxy-(11-beta)-pregn-4-ene-3,20-dione.

Hydrocortisone can also be used in the form of an ester, i.e. esterified in position 17 or 21, e.g. in the form of a butyrate or an acetate, or in the form of a pharmaceutically acceptable salt:

As is known, progesterone is a steroid hormone having formula:

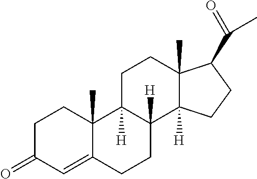

IUPAC name: pregn-4-ene-3,20-dione.

The composition according to the present invention is preferably in the form of a hydroalcoholic solution, i.e. the various active substances are dissolved in a mixture of water and at least one water soluble alcohol, preferably a water:ethanol mixture with an ethanol content of from 60 to 96%.

As an alternative, the composition according to the present invention can be used in another form suitable for a topical application, e.g. in the form of a cream, an emulsion, a gel, etc.

In another aspect, the present invention relates to the use of a composition as defined above for manufacturing a medicament for the treatment of baldness, particularly of androgenetic alopecia.

The composition according to the present invention may further comprise other ingredients, such as e.g.: active ingredients with a complementary and/or integrative action; ingredients able to make transdermal vehiculation easier; cosmetological excipients, flavouring substances, perfumes, colouring agents, stabilizers, glycol, dodecalene (tripropylen glycol citrate), etc.

In particular, the composition according to the present invention may further comprise minoxidil (3-hydroxy-2-imino-6-(1-piperidyl)pyrimidin-4-amine) or a pharmaceutically acceptable salt thereof (e.g. sulphate, hydrochloride), in an amount of from 1 to 10% by weight.

Moreover, the composition according to the present invention can comprise melatonin in an amount of from 0.01 to 0.1% by weight.

The present invention will now be further disclosed with some examples of embodiment, which are provided for a merely illustrative and therefore non-limiting purpose.

### EXAMPLE 1

### Composition for the Treatment of Baldness in Female Patients

The following composition was prepared:

| | |
|---|---|
| hydrocortisone butyrate | 0.05% by weight |
| base estrone | 0.05% by weight. |

The active substances were dissolved in 75% ethyl alcohol.

The action of the aforesaid composition was evaluated on a sample of 200 women suffering from the so-called female androgenetic alopecia.

A double test versus common trichological preparations existing on the market was prepared, in which the composition according to the invention was applied to 100 patients, whereas a commercial solution was applied to other 100 patients. The application was made 3 times a week after head washing on moist hair. After application the lotion was distributed on the scalp and penetration was helped by a light finger massage.

At the end of the test, it was found that in 95% of the patients treated with the composition according to the invention hair loss had stopped and in 60% a remarkable hair re-growth could be observed.

It should be pointed out that no adverse effect occurred even after several months of administration.

### EXAMPLE 2

### Composition for the Treatment of Baldness in Male Patients

The following composition was prepared:

| | |
|---|---|
| progesterone base | 1% by weight |
| hydrocortisone butyrate | 0.05% by weight |
| base estrone | 0.05% by weight. |

The active substances were dissolved in 80% ethyl alcohol.

The action of the aforesaid composition was evaluated on a sample of 200 men suffering from male pattern androgenetic alopecia.

A double test versus common trichological preparations existing on the market was prepared, in which the composition according to the invention was applied to 100 patients, whereas a commercial solution was applied to other 100 patients. The application was made 3 times a week after head washing on moist hair. After application the lotion was distributed on the scalp and penetration was helped by a light finger massage.

At the end of the test, it was found that in 80% of the patients treated with the composition according to the invention hair loss had stopped and in 20% a remarkable hair re-growth could be observed.

It should be pointed out that no adverse effect occurred even after several months of administration.

### EXAMPLE 3

### Comparative Test

A solution of 0.05% by weight of hydrocortisone butyrate and 0.05% by weight of estrone base in 75% ethyl alcohol was prepared. The action of the aforesaid composition was evaluated on a sample of 400 women suffering from the so-called diffused unpatterned alopecia.

A double test versus a commercial solution of 0.1% hydrocortisone butyrate was prepared, in which the above solution was applied to 200 patients, whereas the commercial solution was applied to other 200 patients. The application was made 3 times a week in an amount of 3 ml.

The patients were monitored for 24 months.

After two years, at the end of the test, a significant increase in hair density and thickness was observed in 87% of the patients treated with the solution of hydrocortisone butyrate and estrone, whereas the other patients had benefits in 20% of the cases.

It was not possible to perform a test versus estrone lotion since there is no commercial preparation containing this substance. We made a test with equine conjugated estrogens (consisting of 48% of estrone sulphate, 26% of equilin sulphate, 15% of 17-alpha-dihydroequiline sulphate and small amounts of 17-alpha-estradiol, equilenin, and 17-alpha-dihydroequilenin, all in the form of sodium salts and sulphur esters thereof) in 0.02% solution for topical use, from which positive results were obtained in 30% of the treated cases.

### EXAMPLE 4

### Comparative Test

A solution containing 0.05% by weight of hydrocortisone butyrate, 0.05% by weight of estrone base and 1% by weight of natural progesterone in 75% ethyl alcohol was prepared for treating male androgenetic alopecia. About 200 patients selected for a typical incipient alopecia or for an evident familiar inheritance were treated and monitored for 10 years. At the end of observation time 95% of the treated patients had not developed androgenetic alopecia.

For the treatment of male patients suffering from incipient androgenetic alopecia, progesterone was used in the past with different concentrations of 0.5-1-1.5-2% in hydroalcoholic solution (60-70% ethanol) in a dose of 4 ml pro die. From 1987 to 1990 we had been able to monitor 237 male patients, all of whom were treated with 2.5% progesterone solution (120 mg/die). In these patients we could observe a reduction of premature telogen phases with microscopic examination in 87% of the cases (206 patients), a reduction of telogen percentage with trichogram in 92% of the cases (218 patients), an increase in the number of hair with the trichological count. However, these improvements did not result in practice in any significant aesthetic effect.

1. A composition comprising:
(a) from 0.005 to 0.2% by weight of estrone;
(b) from 0.005 to 0.2% by weight of hydrocortisone or an ester thereof or a pharmaceutically acceptable salt thereof, for use in topical treatment of baldness, particularly of androgenetic alopecia.

2. Composition according to claim 1, comprising:
(a) from 0.01 to 0.1% by weight of estrone;
(b) from 0.01 to 0.1% by weight of hydrocortisone or an ester thereof or a pharmaceutically acceptable salt thereof.

3. Composition according to claim 1, further comprising:
(c) from 0.02 to 6% by weight, preferably from 0.5 to 4% by weight, of progesterone.

4. Composition according to claim 1, wherein hydrocortisone is esterified in position 17 or 21.

5. Composition according to claim 4, wherein hydrocortisone is hydrocortisone butyrate or hydrocortisone acetate.

6. Composition according to claim 1, further comprising minoxidil (3-hydroxy-2-imino-6-(1-piperidyl) pyrimidin-4-amine) or a pharmaceutically acceptable salt thereof, in an amount of from 1 to 10% by weight.

7. Composition according to claim 1, further comprising melatonin in an amount of from 0.01 to 0.1% by weight.

8. Composition according to claim 1, in the form of a hydroalcoholic solution.

9. Composition according to claim 1, in the form of a cream, an emulsion or a gel.

10. Method for topical treatment of baldness, particularly of androgenetic alopecia, which comprises topically administering a composition according to claim 1 to a patient in need thereof.
